(19) United States
(12) Patent Application Publication
(10) Pub. No.: US 2005/0054967 A1
(43) Pub. Date: Mar. 10, 2005

(54) Fome swab applicator for fluid-containing ampule

(76) Inventors: Gary Ashe, Sanborn, NY (US); Edward M. Purizhansky, Williamsville, NY (US)

Correspondence Address: Walter W. Duft, Suite 10, 10255 Main Street, Clarence, NY, 14031, US

(21) Appl. No.: 10/656,849

(22) Filed: Sep. 5, 2003

(51) Int. Cl. A61M 35/00

(52) U.S. Cl. 604/2

(57) ABSTRACT

A foam swab applicator for use in dispensing fluid from a fluid-containing ampule includes an applicator sleeve having a closed base end and an open fluid delivery end, a hermetically sealed, fluid-containing ampule disposed in the applicator sleeve between the base end and the fluid delivery end, and a foam swab delivery tip unit mounted on the applicator sleeve in contacting relationship with. The delivery tip unit may include a delivery tip base member received at the fluid delivery end of the applicator sleeve, and a foam swab element mounted to the delivery tip base member so as to extend away from the applicator sleeve.

### CROSS REFERENCE TO RELATED APPLICATIONS

--Not Applicable--

### BACKGROUND OF THE INVENTION

1. Field of the Invention

The present invention relates to swab applicators for dispensing materials in fluid form from fluid-containing ampules.

2. Description of the Prior Art

By way of background, swab applicators have been used for dispensing fluids of various kind from fluid-containing ampules. FIGS. 1 and 2 illustrate one example in which a swab applicator 2 is used to deliver a topical personal care product 4 in fluid form from a unit dose ampule 6 to the skin of a person (not shown) to be treated with the product. The ampule 6 is commonly formed as a hollow cylindrical housing constructed from either USP type III soda lime glass or USP type I borosilicate glass. The ampule 6 is hermetically sealed and contains a sufficient amount of the product 4 to provide a single recommended dosage unit thereof. Dual chamber ampules (not shown) are also available for delivering two products that must be kept separate until the moment of use.

The prior art swab applicator 2 is formed with an elongated plastic sleeve 8 that is sized to receive the ampule 6 therein. A base end 10 of the sleeve 8 is closed to provide support for the ampule 6. An opposing open end 12 of the sleeve 8 receives a cylindrical cotton swab 14 that is similar in shape and size to a cigarette filter, except that the swab is formed with a rounded fluid delivery tip. The rounded tip end of the cotton swab 14 extends from the sleeve 8 while a base end of the swab is secured within the sleeve in close proximity to one end of the ampule 6.

When it is desired to apply the product 4, the sleeve 8 is squeezed, the ampule 6 ruptures, and the applicator 2 is inverted so that the product is gravity-fed to the cotton swab 14. The cotton swab 14 is placed on the desired area and the product 4 is applied to that location. After the product 4 is exhausted from the ampule 6, the applicator 2 and the empty ampule can be discarded.

It is to improving swab applicators of the foregoing type that the present invention is directed.

### SUMMARY OF THE INVENTION

The foregoing problems are solved and an advance in the art is provided by an improved swab applicator for use in dispensing fluid from a fluid-containing ampule. The swab applicator includes an applicator sleeve having a closed base end and an open fluid delivery end. A hermetically sealed, fluid-containing ampule is disposed in the applicator sleeve between the base end and the fluid delivery end. A foam swab delivery tip unit is mounted on the applicator sleeve, in contacting relationship therewith. The delivery tip unit includes a delivery tip base member received at the fluid delivery end of the applicator sleeve, and a foam swab element mounted to the delivery tip base member so as to extend away from the applicator sleeve

In exemplary implementations of the invention, the foam swab element can be formed from a polymeric foam material, such an open cell polyurethane foam material. It can be generally circular in cross-sectional shape and may include an oblique planar fluid delivery end face. The foam swab element can be bonded or otherwise secured to the delivery tip base member using a suitable interconnection arrangement, such as by way of a recess on a base end of the foam swab element and an annular swab mounting flange on the delivery tip base member that fits into the recess.

In further exemplary implementations of the invention, the delivery tip base member can be provided with a fluid metering aperture that is adapted to meter fluid from the applicator sleeve to the foam swab element. The delivery tip base member can also be formed with a fluid reservoir (accumulator well) that is adapted to receive fluid from the applicator sleeve and maintain the fluid in contact with a base end of the foam swab element. The delivery tip base member may further include a fluid collection barrel that is adapted to be slidably received in the fluid delivery end of the applicator sleeve and to collect fluid from the applicator sleeve. The fluid collection barrel can be formed with a tapered tip to facilitate slideable insertion of the fluid delivery tip into the fluid delivery end of the applicator sleeve.

### BRIEF DESCRIPTION OF THE DRAWING

The foregoing and other features and advantages of the invention will be apparent from the following more particular description of preferred embodiments of the invention, as illustrated in the accompanying Drawing in which:

FIG. 1 is a side view of a prior art cotton swab applicator and associated unit dose ampule containing a topically deliverable personal care product in fluid form;

FIG. 2 is an exploded view of the elements of FIG. 1 showing an applicator sleeve, the ampule, and a cotton swab delivery tip;

FIG. 3 is an oblique partial side/partial bottom view of a foam swab applicator and a fluid-containing ampule constructed in accordance with the present invention;

FIG. 4 is an exploded view of the elements of FIG. 3 showing an applicator sleeve, the ampule, and a foam swab delivery tip unit;

FIG. 5 is a bottom view of the delivery tip unit;

FIG. 6 is a side view of the delivery tip unit;

FIG. 7 is a perspective view of the delivery tip unit;

FIG. 8 is an exploded view of the delivery tip unit showing a foam swab element and a delivery tip base member;

FIG. 9 is a cross-sectional view taken along line 9-9 in FIG. 8;

FIG. 10A is a bottom view of the foam fluid delivery swab element;

FIG. 10B is an end view of the foam swab element looking in the direction of the arrows 10B-10B in FIG. 10A;

FIG. 10C is an end view of the foam swab element looking in the direction of the arrows 10C-10C in FIG. 10A;

FIG. 11A is a bottom view of the delivery tip base member;

FIG. 11B is an end view of the delivery tip base member looking in the direction of the arrows 11B-11B in FIG. 11A; and

FIG. 11C is an end view of the delivery tip base member looking in the direction of the arrows 11C-11C in FIG. 11A.

### DETAILED DESCRIPTION OF PREFERRED EMBODIMENTS

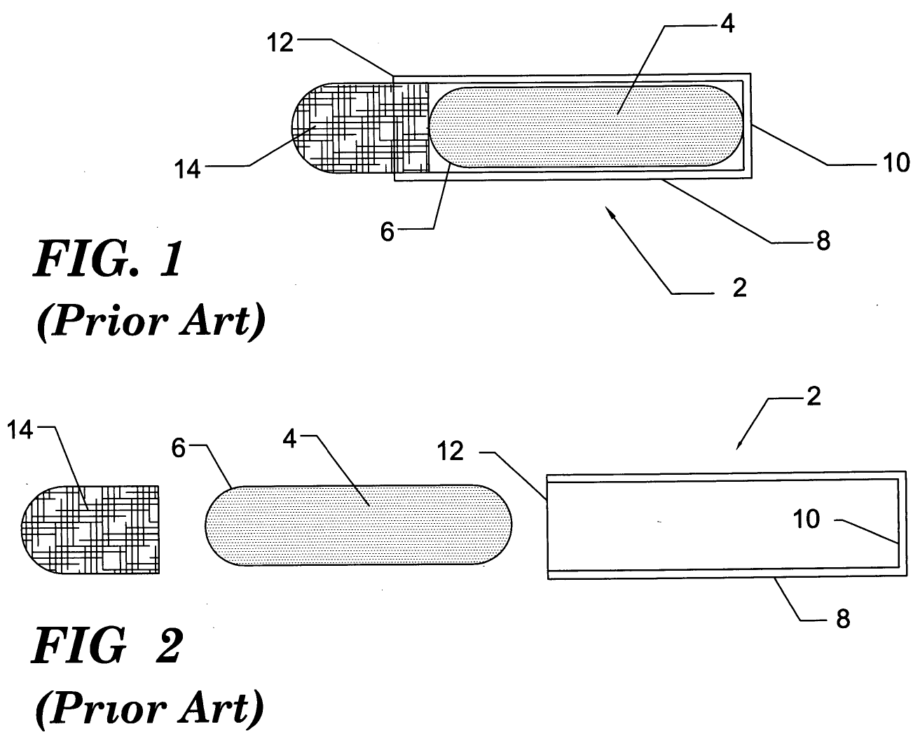

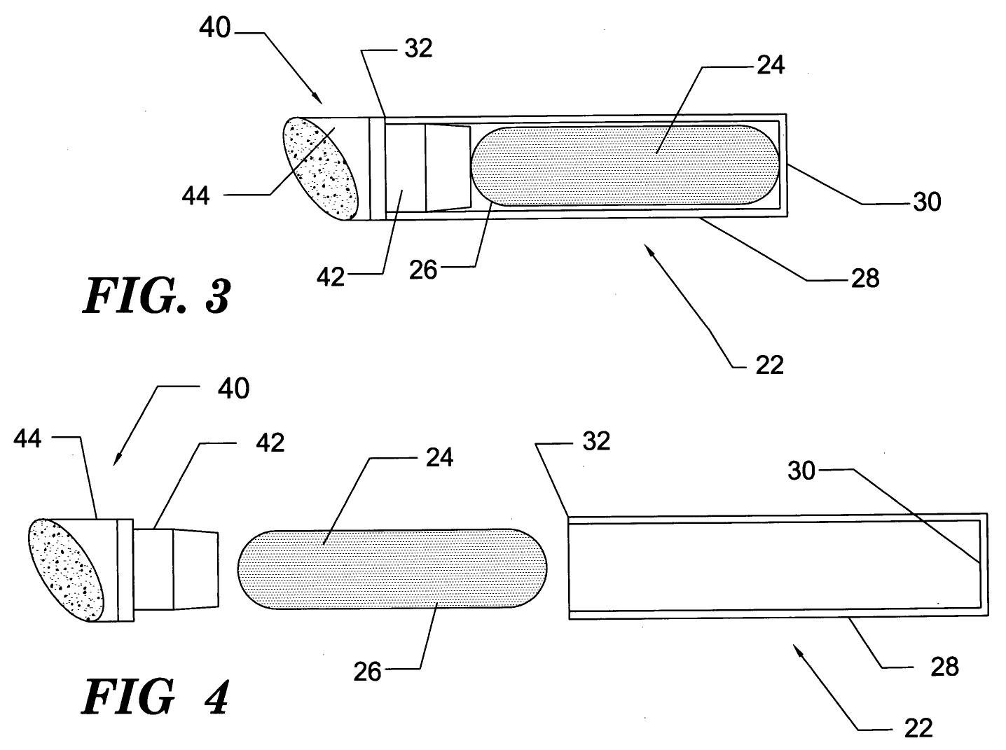

Turning now to the figures, wherein like reference numerals represent like elements in all of the several views, FIGS. 3 and 4 illustrate a foam swab applicator 22 constructed in accordance with one exemplary embodiment of the present invention. The applicator 22 is adapted to deliver a fluid product 24 contained within a hermetically sealed ampule 26. Although the fluid product 24 could be any fluid material to be used for any purpose, it will be assumed for present purposes that the fluid 24 is a topically applied personal care product and that the ampule 26 is sized to deliver a unit dose of the product to an area of a person's skin. More particularly, it will be assumed that the ampule 26 is identical to conventional ampule 6 shown in FIGS. 1 and 2 and described by way of background above.

The swab applicator 22 is shown to include an elongated, generally cylindrical sleeve 28 made from relatively pliable plastic or other suitably flexible material. The sleeve 28 is slightly larger in diameter than the ampule 26. It is formed with a closed base end 30 and an open fluid delivery end 32 (see FIG. 4). The length of the sleeve 28 is selected so that the ampule 26 is disposed between the base end 30 and the fluid delivery end 32 when the swab applicator is assembled, as shown in FIG. 3. Advantageously, the sleeve 28 can be implemented using the sleeve 8 shown in FIGS. 1 and 2 and described by way of background above. Thus, new sleeve constructions are not required in order to practice the present invention.

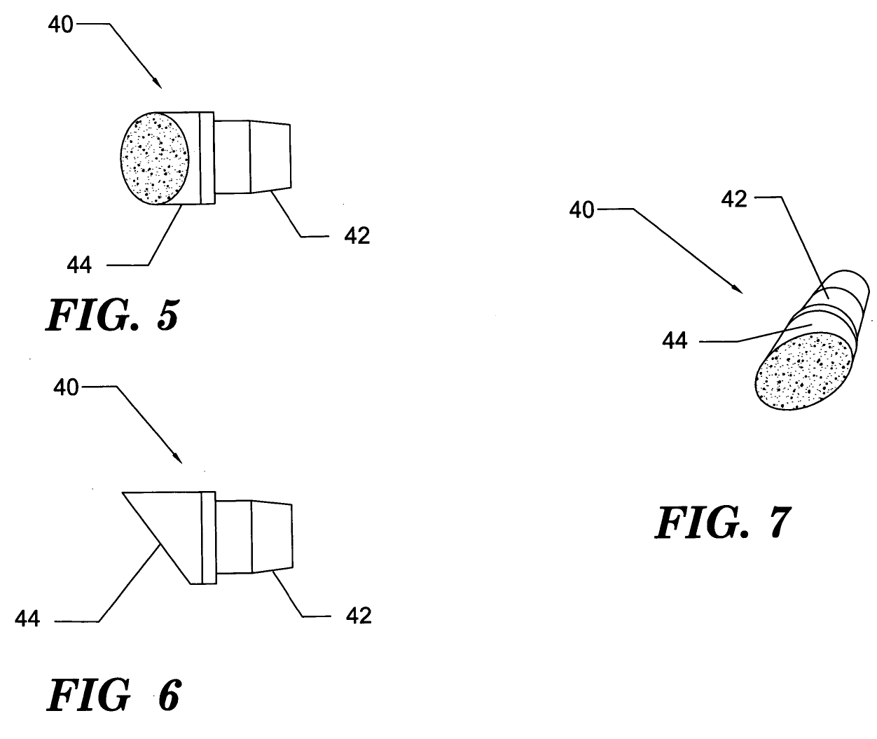

The delivery end 32 of the sleeve 28 receives a foam swab delivery tip unit 40 that is adapted to dispense the product 24 when the ampule 26 is ruptured. The delivery tip unit 40 is mounted on the applicator sleeve 28 so as to be in direct contacting relationship therewith. FIGS. 3 and 4 show the delivery tip unit 40 mounted on the sleeve 28. FIGS. 5, 6, and 7 illustrate the delivery tip unit 40 by itself. These drawing figures set forth an exemplary configuration of the two components that comprise the delivery tip unit 40; namely a delivery tip base member 42 adapted to be received at the fluid delivery end 32 of the applicator sleeve 28, and a foam swab element 44 mounted to the delivery tip base member so as to extend away from the sleeve.

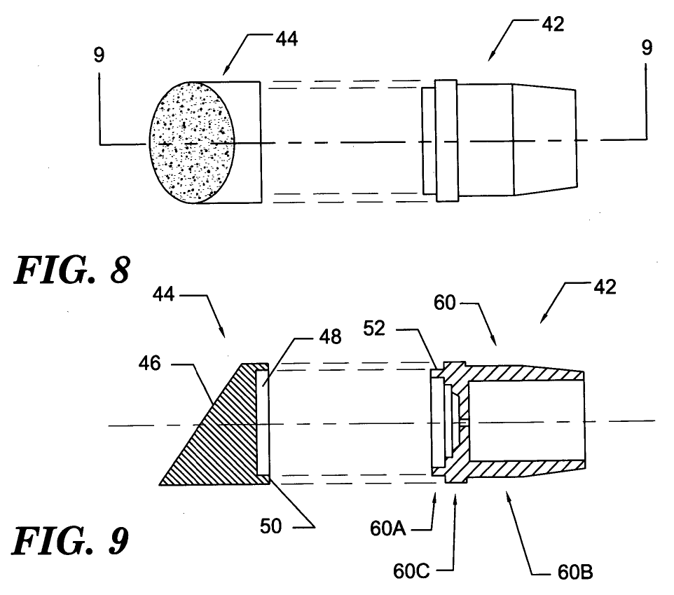

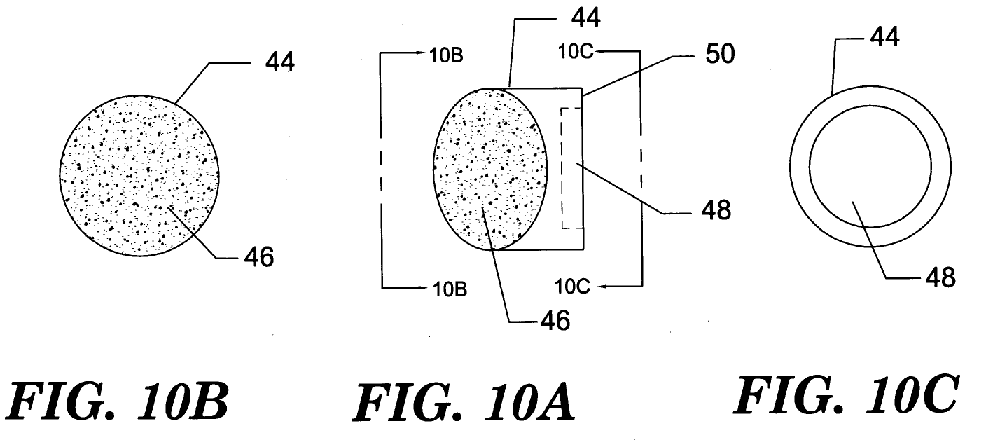

The foam swab element 44 can be formed using any of a variety of resilient polymeric foam materials, such as an open cell polyurethane foam, and can be of any suitable shape and size. In FIGS. 8, 9 and 10A-10C, the foam swab element 44 is shown in one exemplary configuration as being generally circular in cross-sectional shape and having a generally planar fluid delivery end face 46. As further shown in the figures, the fluid delivery end face 46 may be obliquely angled relative to the longitudinal axis of the foam swab element 44. The foam swab element 44 can be bonded or otherwise secured to the delivery tip base member 42 by way of any suitable interconnection arrangement. FIG. 9 shows one exemplary interconnection configuration in which a recess 48 on a base end 50 of the foam swab element 44 receives an annular swab mounting flange 52 formed on the delivery tip base member 42. The recess 48 is shown in FIGS. 9, 10A and 10C as being shaped as a shallow cylindrical well. The recess 48 could also be formed as an annular channel that is sized to receive and engage both the inner and outer walls of the annular swab mounting flange 52. In an alternative interconnection arrangement, the base end 50 of the foam swab element 44 could be formed without the recess 48 and the delivery tip base member 42 could be formed without the annular swab mounting flange 52.

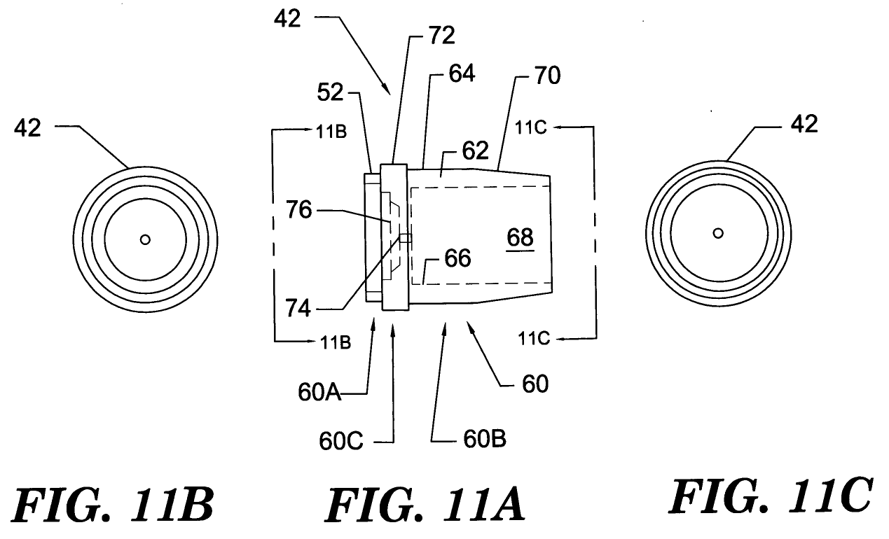

The delivery tip base member 42 can be formed from rigid plastic or other suitable material according to any of a variety of design configurations. In the exemplary configuration shown by way of the drawings, and as best shown in FIGS. 9 and 11A, the delivery tip base member 42 is provided by way of a plug structure 60 that may be thought of as including a first end portion 60A that is adapted to mount to the foam swab element 44, a second end portion 60B that is adapted to mount to the applicator sleeve 2 and a central portion 60C disposed between the two end portions. The first end portion 60A of the plug structure 60 includes the aforementioned annular flange 52. The second end portion 60B of the plug structure 60 includes a fluid collection barrel 62 having a first outer wall portion 64 and an inner wall portion 66. The first outer wall portion 64 is adapted to be slidably received in the fluid delivery end 32 of the applicator sleeve 28. The inner wall portion 66 defines a chamber 68 that collects fluid from the applicator sleeve 28. The fluid collection barrel 62 further includes a tapered tip section defined by a second outer wall portion 70. The second outer wall portion 70 is tapered to facilitate slideable insertion of the plug structure 60 into the fluid delivery end 32 of the applicator sleeve 28.

The central portion 60C of the plug structure 60 includes an outer flange 72 that is adapted on one side to receive the base end 50 of the foam swab element 44 (see FIG. 9). The opposing side of the outer flange 72 is adapted to abut the fluid delivery end 32 of the applicator sleeve 28 (see FIG. 3). The central portion 60C of the plug structure 60 further includes a fluid metering aperture 74 that is adapted to meter fluid from the applicator sleeve 28 to the foam swab element 44. In particular, the fluid metering aperture 74 provides a fluid passage from the chamber 68 of the fluid collection barrel 62 to a stepped fluid reservoir 76 that is adapted to receive fluid from the applicator sleeve 28 and serve as a collection well to maintain the fluid in contact with the base end 50 of the foam swab element 44. In this way, the foam swab element will be kept irrigated with fluid notwithstanding temporary inversion of the swab applicator 22 during use. It will be appreciated that the shape and size of the fluid reservoir 76 can be varied from that shown in FIGS. 9 and 1 IA according to design needs. For example, the reservoir 76 need not be stepped and may be larger or smaller than that shown in FIGS. 9 and 11A.

The swab applicator 22 is used in the same manner as the conventional swab applicator 2 described by way of background above with reference to FIGS. 1 and 2. In particular, a user grasps the applicator sleeve 28 between his or her thumb and fingers. The user squeezes the applicator sleeve 28 until its inner walls place sufficient compressive stress on the ampule 26 to cause it to rupture. This allows the fluid product 24 to escape from the ampule 26 and flow into the applicator sleeve 28. By tilting the applicator sleeve 28 so that its fluid delivery end 32 is lower than its base end 30, the product 24 will flow into the chamber 68 of the plug structure 60, then through the fluid metering aperture 74 into the fluid reservoir 76 until the product comes into contact with the foam swab element 44. The product 24 will then wick through the foam swab element 44 and emerge at the outer surfaces thereof, including the fluid delivery end face 46, such that the product can be applied to a desired location.

Accordingly, an improved foam swab applicator and associated foam swab delivery tip unit have been disclosed. While various embodiments of the invention have been shown and described, it should be apparent that many variations and alternative embodiments could be implemented in accordance with the invention. It is understood, therefore, that the invention is not to be in any way limited except in accordance with the spirit of the appended claims and their equivalents.

1. A foam swab applicator for use in dispensing fluid from a fluid-containing ampule, comprising:
an applicator sleeve having a closed base end and an open fluid delivery end;
a hermetically sealed, fluid-containing ampule disposed in said applicator sleeve between said base end and said fluid delivery end; and
a foam swab delivery tip unit mounted on with said applicator sleeve in contacting relationship therewith, said delivery tip unit including:
a delivery tip base member received at said fluid delivery end of said applicator sleeve; and
a foam swab element mounted to said delivery tip base member so as to extend away from said applicator sleeve.

2. A foam swab applicator in accordance with claim 1 wherein said foam swab element comprises a polymeric foam material.

3. A foam swab applicator in accordance with claim 1 wherein said foam swab element comprises an open cell polyurethane foam material.

4. A foam swab applicator in accordance with claim 1 wherein said foam swab element is generally circular in cross-sectional shape and comprises a planar oblique fluid delivery end face.

5. A foam swab applicator in accordance with claim 1 wherein said foam swab element is bonded to said delivery tip base member.

6. A foam swab applicator in accordance with claim 1 wherein said foam swab element and said delivery tip base member are mounted together by way of a recess on a base end of said foam swab element and an annular swab mounting flange on said delivery tip base member that fits said base end recess.

7. A foam swab applicator in accordance with claim 1 wherein said delivery tip base member comprises a fluid metering aperture that is adapted to meter fluid from said applicator sleeve to said foam swab element.

8. A foam swab applicator in accordance with claim 1 wherein said delivery tip base member comprises a fluid reservoir that is adapted to receive fluid from said applicator sleeve and maintain said fluid in contact with a base end of said foam swab element.

9. A foam swab applicator in accordance with claim 1 wherein said delivery tip base member comprises a fluid collection barrel that is adapted to be slidably received in said fluid delivery end of said applicator sleeve and to collect fluid from said applicator sleeve.

10. A foam swab applicator in accordance with claim 9 wherein said fluid collection barrel comprises a tapered tip to facilitate slideable insertion of said fluid delivery tip into said fluid delivery end of said applicator sleeve.

11. A foam swab delivery tip unit for use in combination with an applicator assembly adapted to dispense fluid, the applicator assembly including an applicator sleeve having a closed base end and an open fluid delivery end, and a hermetically sealed, fluid-containing ampule disposed in the applicator sleeve between the base end and the fluid delivery end, said foam swab delivery tip unit comprising:
a delivery tip base member adapted to be received at and mounted in contacting relationship with the fluid delivery end of the applicator sleeve; and
a foam swab element mounted to said delivery tip base member so as to extend away from the applicator sleeve.

12. A foam swab delivery tip unit in accordance with claim 11 wherein said foam swab element comprises a polymeric foam material.

13. A foam swab delivery tip unit in accordance with claim 11 wherein said foam swab element comprises an open cell polyurethane foam material.

14. A foam swab delivery tip unit in accordance with claim 11 wherein said foam swab element is generally circular in cross-sectional shape and comprises a planar oblique fluid delivery end face.

15. A foam swab delivery tip unit in accordance with claim 111 wherein said foam swab element is bonded to said delivery tip base member.

16. A foam swab delivery tip unit in accordance with claim 11 wherein said foam swab element and said delivery tip base member are mounted together by way of a recess on a base end of said foam swab element and an annular swab mounting flange on said delivery tip base member that fits said base end recess.

17. A foam swab delivery tip unit in accordance with claim 11 wherein said delivery tip base member comprises a fluid metering aperture that is adapted to meter fluid from the applicator sleeve to said foam swab element.

18. A foam swab delivery tip unit in accordance with claim 11 wherein said delivery tip base member comprises a fluid reservoir that is adapted to receive fluid from the applicator sleeve and maintain said fluid in contact with a base end of said foam swab element.

19. A foam swab delivery tip unit in accordance with claim 11 wherein said delivery tip base member comprises a fluid collection barrel that is adapted to be slidably received in the fluid delivery end of the applicator sleeve and to collect fluid from the applicator sleeve.

20. A foam swab delivery tip in accordance with claim 19 wherein said fluid collection barrel comprises a tapered tip to facilitate slideable insertion of said fluid delivery tip into the fluid delivery end of the applicator sleeve.

21. A foam swab applicator for use in dispensing fluid from a fluid-containing ampule, comprising:
an applicator sleeve having a closed base end and an open fluid delivery end;
a hermetically sealed, fluid-containing ampule disposed in said applicator sleeve between said base end and said fluid delivery end; and
a foam swab delivery tip unit mounted on and in contacting relationship with said applicator sleeve, said delivery tip unit including:
a delivery tip base member received at said fluid delivery end of said applicator sleeve; and
a foam swab element mounted to said delivery tip base member so as to extend away from said applicator sleeve;
said swab element comprising an open cell polyurethane foam material and being generally circular in cross-sectional shape and having a planar oblique fluid delivery end face;
said swab element being bonded to said delivery tip base member by way of a recess on a base end of said foam swab element and an annular swab mounting flange on said delivery tip base member that fits said base end recess;
said delivery tip base member including a fluid metering aperture that is adapted to meter fluid from said applicator sleeve to said foam swab element;
said delivery tip base member further including a fluid reservoir that is adapted to receive fluid from said applicator sleeve and maintain said fluid in contact with a base end of said foam swab element; and
said delivery tip base member further including a fluid collection barrel that is adapted to be slidably received in said fluid delivery end of said applicator sleeve and to collect fluid from said applicator sleeve, said fluid collection barrel having a tapered tip to facilitate slideable insertion of said fluid delivery tip into said fluid delivery end of said applicator sleeve.
